(19) United States
(12) Patent Application Publication
(10) Pub. No.: US 2015/0031566 A1
(43) Pub. Date: Jan. 29, 2015

(54) BIOTINYLATED MHC COMPLEXES AND THEIR USES

(71) Applicant: PROIMMUNE LIMITED, Oxford (GB)

(72) Inventors: Catherine Elizabeth NAPPER, WITNEY (GB); Nikolai Franz Gregor SCHWABE, OXFORD (GB)

(21) Appl. No.: 14/340,535

(22) Filed: Jul. 24, 2014

Related U.S. Application Data
(63) Continuation of application No. 11/699,988, filed on Jan. 31, 2007, now abandoned.

(30) Foreign Application Priority Data
Jul. 25, 2006 (GB) GB0614735.9

(51) Int. Cl. G01N 33/569; G01N 33/68; C12P 21/00; C07K 14/74

(52) U.S. Cl. CPC G01N 33/56977; C07K 14/70539; G01N 33/6854; C12P 21/00; C07K 2319/90; G01N 2800/245; G01N 2333/70539; G01N 2440/32
USPC 506/9; 530/394; 435/7.24; 436/501; 435/7.92; 435/68.1; 435/69.6; 435/69.3

(57) ABSTRACT

The invention demonstrates an improved choice of biotinylation peptide to be used in a combination or fusion with an MHC molecule for immobilizing or multimerising such MHC molecules for a variety of purposes.

### FIELD OF THE INVENTION

The invention relates to biotinylated MHC complexes and their uses.

### BACKGROUND

Major Histocompatibility Complex (MHC) molecules, which are found on the cell surface in tissues, play an important role in presenting cellular antigens in the form of short linear peptides to T cells by interacting with T cell receptors (TCRs) present on the surface of T cells. They consist of alpha and beta chains, and a peptide bound in a groove formed by these chains when properly folded.

It has been established that isolated or recombinant forms of MHC-peptide molecules are useful for detecting, separating and manipulating T cells according to the specific peptide antigens these T cells recognise.

European Patent Application EP 812 331 discloses a multimeric binding complex for labeling, detecting and separating mammalian T cells according to their antigen receptor specificity, the complex having the formula (α-β-P)ₙ, wherein (α-β-P) is an MHC peptide molecule, n is ≧2, α comprises an α chain of a MHC I or MHC II class molecule, β comprises a β chain of an MHC protein and P is a substantially homogeneous peptide antigen. The MHC peptide molecule is multimerised by biotinylating the C terminus of one of the α or β chain of the MHC molecule and coupling of MHC monomers to tetravalent streptavidin/avidin or by providing a chimeric protein of an MHC molecule which is modified at the C terminus of one of the α or β chain to comprise an epitope which is recognised by a corresponding antibody that serves as a multimerising entity. The document further teaches use of the MHC oligomers for detecting, labeling and separating specific T cells according to their TCR specificity.

In one embodiment EP812331 describes a biotinylated version of an MHC monomer whereby a biotinylation peptide is fused to the α or β chain of the MHC monomer and which can be biotinylated with a biotinylating enzyme. The sequence for the biotinylation peptide used in an example is disclosed in EP711303.

EP711303 discloses biotinylation peptides of less than 50 amino acids in length and more specifically it discloses a group of mimetic peptides that can be as short as 14 amino acids in length, which mimic the function of naturally occurring biotinylation peptides that are usually significantly longer than 50 amino acids in length. While the short mimetic peptides of EP711303 approximate the conformation of the enzyme recognition site of a natural biotinylation peptide, they do not form the globular domain structure that is typical for naturally occurring biotinylation peptides.

U.S. Pat. No. 5,252,466 describes fusion proteins having a site for in vivo post translational modifications and methods for making and purifying them. More specifically U.S. Pat. No. 5,252,466 discloses fusion proteins, which can be biotinylated on a natural biotinylation domain with a biotinylating enzyme in vivo.

US2003/0017447A1 discloses the use of recombinant biotinylated MHC molecules in the detection of anti-HLA antibodies in serum samples from prospective transplant recipients, whereby anti-HLA antibody activity can be determined against a single MHC allele at a time. In an example US2003/0017447A1 uses monomeric recombinant MHC-peptide complexes for this purpose, which have been made similarly to those of EP812,331 using the short biotinylation peptides of EP711,303 fused to the MHC alpha chain of Class I MHC molecules.

In summary biotinylated MHC-peptide complexes have been used in the past for a variety of purposes. Two main applications include:

1) use of such complexes in forming multimeric and specifically tetrameric MHC-peptide complexes for detecting and separating antigen specific T cells, as seen in EP812331; and
2) use of such complexes in detecting anti-HLA serum antibodies in serum samples from prospective organ recipients, as seen in US2003/0017447.

In both cases in the past biotinylated MHC peptide complexes have been made by fusing a short mimetic biotinylation peptide as described in EP711303 to the C-terminus of the MHC alpha or chain.

The biotinylation peptide can then be biotinylated with a biotinylating enzyme, such as biotin-protein ligase BirA.

### THE INVENTION

The invention demonstrates an improved choice of biotinylation peptide to be used in a combination or fusion with an MHC molecule for immobilizing or multimerising such MHC molecules for a variety of purposes, such as the applications described in EP812331 and US2003/0017447A1.

Although the length of the short mimetic biotinylation peptide was heralded as an advantage over longer naturally occurring sequences it can be advantageous to use a larger biotinylation domain in order to obtain better spacing of the biotinylated residue in the fusion protein from the MHC molecule while maintaining a rigid and defined structure of the folded protein complex.

We have shown for the first time that the fusion between an MHC complex and a naturally occurring biotinylation domain larger than 50 amino acids, such as the biotinyl carboxyl carrier protein (BCCP) subunit of acetyl CoA carboxylase in E. coli, can be used to generate biotinylated MHC complexes. Such complexes and multimers thereof are useful for detecting antigen specific T cells in flow cytometry and also for detecting anti-HLA serum antibodies in the sera of transplant recipients. In this transplantation context it is believed that the extra spacing between the biotinylation site of the fused MHC complexes of the invention and the functional MHC portion of such complexes can provide an improvement of the recognition of certain epitopes located on the functional MHC portion by anti-MHC antibodies.

We have found that the biotinylated MHC complexes can be generated easily with known synthesis methods, be produced at good yields compared to the complexes of the prior art and be biotinylated with high efficiency.

In its first aspect thereof the present invention comprises a chimeric peptide comprising an MHC peptide and a biotinylation peptide which either is a natural biotinylation peptide or has a greater than 70% sequence homology to a natural biotinylation peptide.

In its second aspect thereof the present invention comprises a chimeric peptide comprising an MHC peptide and a biotinylation peptide wherein the biotinylation peptide has a minimal sequence required for being biotinylated that is longer than 50 amino acids in length.

In one embodiment the MHC peptide is a Class I MHC peptide.

In an alternative embodiment the MHC peptides is a Class II MHC peptide.

In a further embodiment the biotinylation peptide is biotinylated.

Preferably the biotinylation peptide is located in the chimeric protein after the C-terminal end of the MHC peptide.

In one embodiment the MHC peptide and the biotinylation peptide are separated from one another by a linker sequence.

In one embodiment the biotinylation peptide is selected from the group consisting of the biotinylation domain of BCCP and Proprionibacterium shermanii 1.3S subunit of transcarboxilase.

In a third aspect thereof the present invention comprises an MHC peptide complex with the formula (α-β-P), wherein α comprises an α chain of a MHC I or MHC II class molecule, β comprises an β chain of a MHC I or MHC II class molecule, and P is a peptide antigen bound in the binding groove of the MHC molecule, wherein said MHC peptide complex comprises a chimeric protein of the invention.

In one embodiment peptide antigen P bound in the binding groove is substantially homogeneous.

In a fourth aspect thereof the present invention comprises a multimeric binding complex having the formula (α-β-P)ₙ, wherein (α-β-P) is an MHC peptide complex of the present invention, and wherein n≧2.

In one embodiment n=4.

In one embodiment the MHC peptide complexes are biotinylated and the multimeric binding complex is formed by binding the biotinylated MHC peptide complexes to a multivalent entity that binds to biotin with high affinity.

In this context “high affinity” means that the binding interaction typically subsists for more than 30 minutes and preferably for several hours.

In a further embodiment the multivalent entity is an avidin family protein and more preferably streptavidin.

In yet a further embodiment the multimeric binding complex comprises a label.

In a fifth aspect thereof the present invention comprises a method of labelling and or detecting mammalian T cells according to the specificity of their antigen receptor, the method comprising combining a multimeric binding complex according to the invention and a suspension or biological sample comprising T cells, and detecting the presence of specific binding of said complex and at least one of the T cells.

In a sixth aspect thereof the present invention comprises a method of separating mammalian T cells according to the specificity of their antigen receptor, the method comprising combining a multimeric binding complex according to the invention and a suspension or biological sample comprising T cells, and separating one or more T cells bound to said complex from unbound cells.

In a seventh aspect thereof the present invention comprises a method of detecting the presence of one or more anti-MHC antibodies in a sample comprising contacting said sample with at least one MHC complex of the invention or at least one multimeric binding complex of the invention and detecting the binding or absence of binding of the one or more anti-MHC antibodies to either said MHC complex(es) or multimeric binding complex(es).

In one embodiment the antibodies which are detected are IgG, IgM or IgA.

In another embodiment the peptide antigen P is derived from an antigen that occurs in less than 5% of a population group.

“Population group” in this context shall mean a group of individuals from the general population, which may or may not be restricted to a specific region, which may be subjected to testing of fluid or tissue samples with one or more of the MHC complexes, multimeric binding complexes and/or methods of the invention.

In another embodiment the MHC complex(es) is(are) attached to a solid support.

In another embodiment the MHC complex(es) is(are) biotinylated and immobilized to the support through binding to an avidin family protein which is itself bound to the solid support.

In another embodiment the avidin family protein is streptavidin.

In another embodiment said solid support is a spherical bead.

In another embodiment the bead comprises a detectable label.

In an alternative embodiment the solid support is a nitrocellulose strip.

In another alternative embodiment the solid support is an ELISA plate.

In one embodiment the MHC complex(es) is(are) synthesized in a prokaryotic expression system.

In one embodiment the sample is a body fluid sample.

In one embodiment the bound antibody or absence thereof is detected via an immunosorbent assay using an antibody conjugated to a signalling means.

In one embodiment a single solid support is carrying two or more different ones of the MHC complexes or of the multimeric binding complexes at discrete locations on said solid support.

In an alternative embodiment two or more different ones of the MHC complexes or of the multimeric binding complexes are immobilized on different ones of said solid supports.

In an eighth aspect thereof the present invention comprises a method for determining the suitability of an organ to be transplanted for a transplant recipient, comprising the method of the seventh aspect of the invention, wherein the sample is a serum sample of the prospective transplant recipient and the presence of antibodies in the recipient that are reactive to at least one MHC molecule in the organ are detected and at least one MHC allele is determined against which such antibodies are reactive.

In a ninth aspect thereof the present invention comprises a method for determining a rejection reaction against a transplanted organ comprising the method the seventh aspect of the invention, wherein the sample is a serum sample of the transplant recipient by detecting the presence of antibodies in the recipient that are reactive to at least one MHC molecule in the organ are detected and at least one MHC allele is determined against which such antibodies are reactive

In a tenth aspect thereof the invention comprises a method of depleting a sample of anti-MHC molecule antibodies comprising at least the steps of contacting said sample with at least one MHC complex of the invention or at least one multimeric binding complex of the invention optionally attached to a solid support, and removing at least the MHC complex(es) or multimeric binding complexes from the sample to which at least one anti-MHC antibody contained within the sample has bound.

In a eleventh aspect thereof the invention comprises a kit comprising at least the following components: a) one or more recombinant MHC complexes of the invention or at least one multimeric binding complex of the invention; b) optionally a solid support, together with means for attachment of the MHC complex(es) or the multimeric binding complex(es); and c) a means for detecting anti-MHC-antibodies, preferably an antibody which binds to the complex formed between said MHC complex(es) or multimeric binding complex(es) and naturally occurring antibodies to said molecules.

In a twelfth embodiment thereof the invention comprises methods for biotinylating a chimeric peptide as follows:

One embodiment comprises a method for biotinylating a chimeric peptide, the chimeric peptide comprising an MHC peptide and a biotinylation peptide which either is a natural biotinylation peptide or has a greater than 70% sequence homology to a natural biotinylation peptide wherein the chimeric peptide is incubated in a reaction mixture comprising biotin or a biotin analogue and a biotinylating enzyme, resulting in the biotinylation of the chimeric peptide.

Another embodiment comprises a method for biotinylating a chimeric peptide, the chimeric peptide comprising an MHC peptide and a biotinylation peptide which is biotinylation peptide is either is a natural biotinylation peptide or has a greater than 70% sequence homology to a natural biotinylation peptide the method comprising (i) constructing a recombinant DNA expression vector that encodes the chimeric peptide, (ii) transforming a recombinant host cell with said vector, and (iii) culturing said host cell in the presence of biotin or a biotin analogue and under conditions such that said fusion protein and a biotinylating enzyme are expressed, resulting in the biotinylation of said chimeric peptide.

Another embodiment comprises a method for biotinylating a chimeric peptide, the chimeric peptide comprising an MHC peptide and a biotinylation peptide which biotinylation peptide has a minimal sequence required for being biotinylated that is longer than 50 amino acids in length wherein the chimeric peptide is incubated in a reaction mixture comprising biotin or a biotin analogue and a biotinylating enzyme, resulting in the biotinylation of the chimeric peptide.

Another embodiment comprises a method for biotinylating a chimeric peptide, the chimeric peptide comprising an MHC peptide and a biotinylation peptide which biotinylation peptide has a minimal sequence required for being biotinylated that is longer than 50 amino acids in length the method comprising (i) constructing a recombinant DNA expression vector that encodes the chimeric peptide, (ii) transforming a recombinant host cell with said vector, and (iii) culturing said host cell in the presence of biotin or a biotin analogue and under conditions such that said fusion protein and a biotinylating enzyme are expressed, resulting in the biotinylation of said chimeric peptide.

The functional monomeric MHC complexes of the invention will usually be soluble isolated or recombinant MHC complexes that may be derived from MHC class I or class II complexes, preferably the extra-cellular part of an MHC class I complex or the extra-cellular part of an MHC class II complex. Each of these complexes consists of an MHC α chain and an MHC β chain. The functional complex(es) may further comprise a peptide antigen P bound in the binding groove formed by the MHC α and β chains.

The MHC complexes may be from any vertebrate species, e.g. primate species, particularly humans; rodents, including mice, rats, hamsters, and rabbits; equines, bovines, canines, felines; etc. Of particular interest are the human HLA proteins, and the murine H-2 proteins. Included in the HLA proteins are the class II subunits HLA-DPα, HLA-DPβ, HLA-DQα, HLA-DQβ, HLA-DRα and HLA-DRβ, and the class I proteins HLA-A, HLA-B, HLA-C, and β2-microglobulin. Included in the murine H-2 subunits are the class I H-2K, H-2D, H-2L, and the class II I-Aα, I-Aβ, I-Eα and I-Eβ, and β2-microglobulin. Amino acid sequences of some representative MHC proteins are referenced in EP 812 331. In one embodiment the MHC complexes will be from classical MHC molecules, including from classical Class I and Class II MHC molecules. Also included in the scope of this invention are non-classical examples such as HLA-E, HLA-F, HLA-G, Qa1, and CD1. The CD1 monomer may instead of the peptide have a lipid bound in its binding groove. The present invention is also applicable to the situation where a lipid instead of a peptide is bound and the skilled worker will be capable of translating the above oligomerisation protocols to this situation.

In a preferred embodiment, the MHC peptide chains correspond to the soluble form of the normally membrane-bound protein. For class I subunits, the soluble form is derived from the native form by deletion of the transmembrane and cytoplasmic domains. For class I proteins, the soluble form will include the α1, α2 and α3 domains of the α chain. For class II proteins the soluble form will include the α1 and α2 or β1 and β2 domains of the α chain or β chain, respectively.

Not more than about 10, usually not more than about 5, preferably none of the amino acids of the transmembrane domain will be included. The deletion may extend as much as about 10 amino acids into the α3 domain. Preferably none of the amino acids of the α3 domain will be deleted. The deletion will be such that it does not interfere with the ability of the α3 domain to fold into a functional disulfide bonded structure. The class I β chain, β2m, lacks a transmembrane domain in its native form, and does not have to be truncated. Generally, no class II subunits will be used in conjunction with class I subunits.

The above deletion is likewise applicable to class II subunits. It may extend as much as about 10 amino acids into the α2 or β2 domain, preferably none of the amino acids of the α2 or β2 domain will be deleted. The deletion will be such that it does not interfere with the ability of the α2 or β2 domain to fold into a functional disulfide bonded structure. In addition, one may wish to substitute one or more amino acids with a different amino acid for similar reasons, usually not substituting more than about five amino acids in any one domain.

A natural biotinylation peptide within the scope of the present invention means a peptide derived from a biotinylation domain, that is occurring naturally in an organism and that is capable of being biotinylated by a corresponding biotinylating enzyme. The biotinylation peptide will be derived in a manner such that it retains its property of being biotinylated by a corresponding biotinylating enzyme and have a greater than 70%, preferably greater than 80%, and more preferably greater than 90% sequence homology with a segment of the naturally occurring biotinylation domain that is also still capable of being biotinylated by a corresponding biotinylating enzyme. For example the biotinylation peptide may be derived by selecting the minimal sequence from the naturally occurring biotinylation domain that can be biotinylated by a corresponding enzyme.

Examples for the natural biotinylation peptide include the biotinyl carboxyl carrier protein (BCCP) subunit of acetyl CoA carboxylase. The corresponding biotinylation reaction is catalyzed by the biotin-protein ligase (BirA), the product of the birA gene (U.S. Pat. No. 5,252,466). Another example for a natural biotinylation peptide is the 1.3S subunit of transcarboxilase in Proprionibacterium shermanii.

All natural biotinylation domains known to date have minimally required sequences for being biotinylated by a biotinylating enzyme that are longer than 50 amino acids in length (EP711303).

Other suitable natural biotinylation peptides can be derived from carboxylase proteins that are capable of being biotinylated from such species as, e.g., Homo sapiens, Chicken, E. coli, Tomato, S. cerevisiae.

Typically the minimal or near minimal sequence required for successful enzymatic biotinylation of such proteins would be determined and used in constructing the chimeric peptides of the invention.

The biotinylation peptides of the invention can be fused to any of the termini of the MHC alpha or beta chains, but preferably such a fusion will be made on the C-terminus of one of the chains.

A flexible or rigid linker sequence may be interposed between the MHC peptide and the natural biotinylation peptide in the chimeric protein of the invention to allow for the formation of monomeric MHC complexes that have desired structural properties. Flexible linkers include glycine-serine linkers and rigid linkers include protein sequences that form soluble alpha helical conformations.

The chimeric protein of the invention may further comprise additional amino acid sequences that enable labelling or purification on either one of its polypeptide termini. Equally the MHC complexes of the invention may have such labelling or purification sequences on either one of the termini of the MHC peptide chain which is not a chimeric peptide of the invention.

In the past it has been mentioned that natural biotinylation domains are subject to enzymatic degradation, e.g. during soluble or periplasmic expression. The inventors have found that this issue can be resolved by expressing the fusion proteins in a prokaryotic expression system in inclusion bodies, which reduces protease contamination. The resulting protein denatured can then be purified in a number of repeated wash steps using a detergent wash buffer.

Functional MHC complexes can then be formed by refolding of the complexes using methods well known in the art. It was found that this process ensures that the fused domain stays in tact, especially after the complexes of the invention have undergone proper chromatographic purification.

Where the biotinylation of the chimeric protein is carried out during or following expression of the chimeric protein and during cell culture, expression and culture methods analogous to those described in EP711303 or U.S. Pat. No. 5,252,466 can be followed to achieve biotinylation.

### EXAMPLE

A chimeric protein (SEQ ID NO: 1) is generated by fusing (in N-terminal to C-terminal direction) (i) amino acids 25-300 of the unprocessed HLA-A*0201 alpha (heavy) chain precursor protein (SEQ ID NO: 2) followed by (ii) one glycine and one serine residue followed by (iii) amino acids 81-156 unprocessed precursor of the BCCP protein of acetyl-CoA carboxylase of E. coli strain K12 (SEQ ID NO: 3). The fusion protein is generated using molecular cloning techniques well known in the art. Purified HLA heavy chain can be obtained by expression in inclusion bodies in E. coli. using a suitable expression system, such as the pET system (Novagen, Milwaukee, Wis., USA). Recombinant biotinylated MHC peptide complexes can be generated according to US2003/0017447A1. Specifically, native HLA-A2 monomeric MHC peptide complexes are refolded from denatured MHC alpha and human beta-2-microglobulin in the presence of the peptide GLCTLVAML (EBV BMLF-1 280-288; SEQ ID NO: 4). This peptide is known to bind strongly to HLA-A2 and is an immunodominant T cell epitope from Epstein Barr Virus (EBV). Refolded complexes are biotinylated overnight with the enzyme BirA as described in US2003/0017447A1. Biotinylated complexes are purified by size exclusion chromatography and the ˜50 kD peak is recovered. The recovered material may be subjected to a second chromatography step, such as ion exchange chromatography, if desired, or may simply be concentrated to a suitable protein concentration appropriate for further use. The protein concentration is determined by the method of Bradford and the level of biotinylation can be determined, e.g. via the EZ™ Biotin Quantitation Kit (Pierce Biotechnology, Inc., Rockford, Ill., USA).

For use in detecting antigen specific T cells, the biotinylated MHC complexes of the invention can be conjugated in a 4:1 molar ration to fluorescent labelled streptavidin, such as streptavidin:PE (Molecular Probes, Eugene, Oreg., USA). The complexes can then be used e.g. in flow cytometry to detect antigen-specific T cells as described in EP812331.

Similarly, for use in detecting anti-HLA antibodies the complexes can be plated into streptavidin-coated ELISA plates using a single-specificity HLA molecule per well (HLA complexes of a single allele in one well, HLA complexes of different alleles in different wells).

A typical ELISA set up would be as follows:

Coat wells with 100 μl Streptavidin (1 ng/μl; 1:1000 dilution of 1 mg/ml stock) in Coating buffer (0.1 M NaHCO₃, pH8.3) and leave at 4° C. overnight. Wash plates 4 times with phosphate buffered saline (PBS)-Tween® (0.1%). Add 200 μl of Blocking buffer (5% bovine serum albumin (BSA)/PBS+5% Glycine) and leave at room temperature for 1 hour. Wash plates 4×PBS-Tween® (0.1%). Add 100 μl MHC monomers (0.5 ng/μl) in coating buffer to each well (single MHC allele per well) and leave at room temperature for 1 hour. Wash plates 4×PBS-Tween® (0.1%). Add 50 μl of serum (1:10 dilution) in PBS to each well and leave at room temperature for 1 hour. Wash plates 4×PBS-Tween® (0.1%). Add 100 μl Rabbit anti Human IgA, G, M, kappa, lambda-horseradish peroxidase (HRP) (1:5000 dilution) in PBS and incubate at room temperature on the shaker at 250 rpm for 1 hour. Wash plates 4×PBS-Tween® (0.1%). Add 50 μl tetramethylbenzidine (TMB) and leave it for 10 minutes. Stop reaction with 50 μl H₂SO₄ and measure OD₄₅₀.

APPENDIX
SEQUENCE LISTING

SEQ ID NO: 1
A2BCCP Chimeric Protein
Origin: Artificial

| | |
|---|---|
| 1 | GSHSMRYFFT SVSRPGRGEP RFIAVGYVDD TQFVRFDSDA ASQRMEPRAP WIEQEGPEYW |
| 61 | DGETRKVKAH SQTHRVDLGT LRGYYNQSEA GSHTVQRMYG CDVGSDWRFL RGYHQYAYDG |
| 121 | KDYIALKEDL RSWTAADMAA QTTKHKWEAA HVAEQLRAYL EGTCVEWLRR YLENGKETLQ |
| 181 | RTDAPKTHMT HHAVSDHEAT LRCWALSFYP AEITLTWQRD GEDQTQDTEL VETRPAGDGT |
| 241 | FQKWAAVVVP SGQEQRYTCH VQHEGLPKPL TLRWEPGSHI VRSPMVGTFY RTPSPDAKAF |
| 301 | IEVGQKVNVG DTLCIVEAMK MMNQIEADKS GTVKAILVES GQPVEFDEPL VVIE |

SEQ ID NO: 2
HLA class I histcompatibility antigen, A-2 alpha chain precursor
Origin: Human

| | |
|---|---|
| 1 | MAVMAPRTLV LLLSGALALT QTWAGSHSMR YFFTSVSRPG RGEPRFIAVG YVDDTQFVRF |
| 61 | DGDAASQRME PRAPWIEQEG PEYWDGETRK VKAHSQTHRV DLGTLRGYYN QSEAGSHTVQ |
| 121 | RMYGCDVGSD WRFLRGYHQY AYDGKDYIAL KEDLRSWTAA DMAAQTTKHK WEAAHVAEQL |
| 181 | RAYLEGTCVE WLRRYLENGK ETLQRTDAPK THMTHHAVSD HEATLRCWAL SFYPAEITLT |
| 241 | WQRDGEDQTQ DTELVETRPA GDGTFQKWAA VVVPSGQEQR YTCHVQHEGL PKPLTLRWEP |
| 301 | SSQPTIPIVG IIAGLVLFGA VITGAVVAAV MWRRKSSDRK GGSYSQAASS DSAQGSDVSL |
| 361 | TACKV |

SEQ ID NO: 3
Biotin carboxyl carrier protein of acetyl-CoA carboxylase
Origin E. coli, strain K12

| | |
|---|---|
| 1 | MDIRKIKKLI ELVEESGISE LEISEGEESV RISRAAPAAS FPVMQQAYAA PMMQQPAQSN |
| 61 | AAAPATVPSM EAPAAAEISG HIVRSPMVGT FYRTPSPDAK AFIEVGQKVN VGDTLCIVEA |
| 121 | MKMMNQIEAD KSGTVKAILV ESGQFVEFDE PLVVIE |

SEQ ID NO: 4
Epstein Barr Virus BMLF-1 protein amino acids 280-288
Origin: Epstein Barr Virus

| | |
|---|---|
| 1 | GLCTLVAML |

1. A chimeric peptide comprising an MHC peptide and a biotinylation peptide which either is a natural biotinylation peptide or has a greater than 70% sequence homology to a natural biotinylation peptide, the biotinylation peptide comprising a minimal sequence from a naturally occurring biotinylation domain that can be biotinylated by a corresponding enzyme.

2. The chimeric peptide comprising an MHC peptide and a biotinylation peptide wherein the biotinylation peptide has a minimal sequence required for being biotinylated that is longer than 50 amino acids in length the biotinylation peptide comprising a minimal sequence from a naturally occurring biotinylation domain that can be biotinylated by a corresponding enzyme.

3. The chimeric peptide of claim 1 wherein the MHC peptide is a Class I MHC peptide or a Class II MHC peptide.

4. The chimeric peptide of claim 2 wherein the MHC peptide is a Class I MHC or a Class II MHC peptide.

5. The chimeric peptide of claim 1 wherein the biotinylation peptide is biotinylated.

6. The chimeric peptide of claim 1 wherein the biotinylation peptide is located in the chimeric protein after the C-terminal end of the MHC peptide.

7. The chimeric peptide of claim 1 wherein the MHC peptide and the biotinylation peptide are separated by a linker sequence.

8. The chimeric peptide of claim 1 wherein the biotinylation peptide is selected from the group consisting of the biotinylation domain of BCCP and Proprionibacterium shermanii 1.3S subunit of transcarboxilase.

9. An MHC peptide complex with the formula (α-β-P), wherein α comprises an α chain of a MHC I or MHC II class molecule, β comprises an β chain of a MHC I or MHC II class molecule, and P is a peptide antigen bound in the binding groove of the MHC molecule, wherein said MHC peptide complex comprises a chimeric peptide of claim 1.

10. The MHC peptide complex of claim 9, wherein the peptide antigen P bound in the groove is substantially homogeneous.

11. An Multimeric binding complex having the formula (α-β-P)ₙ, wherein (α-β-P) is the MHC peptide complex of claim 9, and wherein n≧2.

12. The multimeric binding complex of claim 11 wherein the MHC peptide complexes are biotinylated and the multimeric binding complex is formed by binding the biotinylated MHC peptide complexes to a multivalent entity that binds to biotin with high affinity.

13. The multimeric binding complex of claim 12 wherein the multivalent entity is an avidin family protein.

14. The multimeric binding complex of claim 11 comprising a label.

15. A method of labelling and or detecting mammalian T cells according to the specificity of their antigen receptor, the method comprising
(i) combining a multimeric binding complex according claim 11 and a suspension or biological sample comprising T cells, and
(ii) detecting the presence of specific binding of said complex and at least one of the T cells.

16. A method of separating mammalian T cells according to the specificity of their antigen receptor, the method comprising
(i) combining a multimeric binding complex according to claim 11 and a suspension or biological sample comprising T cells, and
(ii) separating one or more T cells bound to said complex from unbound cells.

17. A method of detecting the presence of one or more anti-MHC antibodies in a sample comprising contacting said sample with at least one MHC complex according to any of claim 9 and detecting the binding or absence of binding of the one or more anti-MHC antibodies to either the MHC complex(es).

18. The method according to claim 17 wherein the antibodies which are detected are IgG, IgM, or IgA.

19. The method according to claim 17 wherein the peptide antigen P is derived from an antigen that occurs in less than 5% of a population group.

20. The method according to any of claim 17 wherein the MHC complex(es) is(are) attached to a solid support.

21. The method according to claim 20 wherein the MHC complex(es) is(are) biotinylated and immobilized to the solid support through binding to an avidin family protein which is itself bound to the solid support.

22. The method of claim 21 wherein the avidin family protein is streptavidin.

23. The method according to claim 20 wherein said solid support is a spherical bead.

24. The method according to claim 23 wherein bead comprises a detectable label.

25. The method according to claim 20 wherein said solid support is a nitrocellulose strip.

26. The method according to claim 20 wherein said solid support is an ELISA plate.

27. The method according to claim 17 wherein the MHC complex(es) is(are) synthesized in a prokaryotic expression system.

28. The method according to claim 17 wherein the sample is a body fluid sample.

29. The method according to claim 17 wherein the bound antibody or absence thereof is detected via an immunosorbent assay using an antibody conjugated to a signaling means.

30. The method according to claim 17 wherein a single solid support is carrying two or more different ones of the MHC complexes or of the multimeric binding complexes at discrete locations on said solid support.

31. The method according to claim 17 wherein two or more different ones of the MHC complexes or of the multimeric binding complexes are immobilized on a different ones of said solid supports.

32. A method for determining the suitability of an organ to be transplanted for a transplant recipient, comprising the method of claim 17, wherein the sample is a serum sample of the prospective transplant recipient and the presence of antibodies in the recipient that are reactive to at least one MHC molecule in the organ are detected and at least one MHC allele is determined against which such antibodies are reactive.

33. A method for determining a rejection reaction against a transplanted organ comprising the method of claim 17, wherein the sample is a serum sample of the transplant recipient by detecting the presence of antibodies in the recipient that are reactive to at least one MHC molecule in the organ are detected and at least one MHC allele is determined against which such antibodies are reactive.

34. A method of depleting a sample of anti-MHC molecule antibodies comprising at least the steps of contacting said sample with at least one MHC complex of any of claim 9, optionally attached to a solid support, and removing at least the MHC complex from the sample to which at least one anti-MHC antibody contained within the sample has bound.

35. A kit comprising at least the following components: a) one or more recombinant MHC complexes according to claim 9; b) optionally a solid support, together with means for attachment of the MHC complex(es); and c) a means for detecting anti-MHC-antibodies.

36. A method for biotinylating a chimeric peptide, the chimeric peptide comprising an MHC peptide and a biotinylation peptide which either is a natural biotinylation peptide or has a greater than 70% sequence homology to a natural biotinylation peptide wherein the chimeric peptide is incubated in a reaction mixture comprising biotin or a biotin analogue and a biotinylating enzyme, resulting in the biotinylation of the chimeric peptide.

37. A method for biotinylating a chimeric peptide, the chimeric peptide comprising an MHC peptide and a biotinylation peptide which is biotinylation peptide is either is a natural biotinylation peptide or has a greater than 70% sequence homology to a natural biotinylation peptide the method comprising (i) constructing a recombinant DNA expression vector that encodes the chimeric peptide, (ii) transforming a recombinant host cell with said vector, and (iii) culturing said host cell in the presence of biotin or a biotin analogue and under conditions such that said fusion protein and a biotinylating enzyme are expressed, resulting in the biotinylation of said chimeric peptide.

38. A method for biotinylating a chimeric peptide, the chimeric peptide comprising an MHC peptide and a biotinylation peptide which biotinylation peptide has a minimal sequence required for being biotinylated that is longer than 50 amino acids in length wherein the chimeric peptide is incubated in a reaction mixture comprising biotin or a biotin analogue and a biotinylating enzyme, resulting in the biotinylation of the chimeric peptide.

39. A method for biotinylating a chimeric peptide, the chimeric peptide comprising an MHC peptide and a biotinylation peptide which biotinylation peptide has a minimal sequence required for being biotinylated that is longer than 50 amino acids in length the method comprising (i) constructing a recombinant DNA expression vector that encodes the chimeric peptide, (ii) transforming a recombinant host cell with said vector, and (iii) culturing said host cell in the presence of biotin or a biotin analogue and under conditions such that said fusion protein and a biotinylating enzyme are expressed, resulting in the biotinylation of said chimeric peptide.


SEQUENCE LISTING (4 sequences)

SEQ ID NO: 1 - protein, 354 aa, Artificial Sequence [sequence omitted]

SEQ ID NO: 2 - protein, 365 aa, Human [sequence omitted]

SEQ ID NO: 3 - protein, 156 aa, E. coli, strain K12
MDIRKIKKLIELVEESGISELEISEGEESVRISRAAPAASFPVMQQAYAAPMMQQPAQSNAAAPATVPSMEAPAAAEISGHIVRSPMVGTFYRTPSPDAKAFIEVGQKVNVGDTLCIVEAMKMMNQIEADKSGTVKAILVESGQPVEFDEPLVVIE

SEQ ID NO: 4 - protein, 9 aa, Epstein Barr Virus
GLCTLVAML